(19) United States
(12) Patent Application Publication
(10) Pub. No.: US 2026/0224674 A1
(43) Pub. Date: Aug. 6, 2026

(54) COMPOSITIONS COMPRISING PEDF-DERIVED SHORT PEPTIDES AND MESENCHYMAL STEM CELLS FOR BONE/CARTILAGE REGENERATION

(71) Applicant: BRIM Biotechnology, Inc., Taipei (TW)

(72) Inventors: Frank Wen-Chi LEE, Orlando, FL (US); Huei-Ling CHANG, Taipei (TW); Ya-Lang HUANG, Taipei (TW); Chih-Te CHIEN, Taipei (TW); Sin-Tzu NING, Taipei (TW); Chih-Hung CHANG, Taipei (TW); Hsiu-Jung LIAO, Taipei (TW)

(73) Assignee: BRIM Biotechnology, Inc., Taipei (TW)

(21) Appl. No.: 19/119,097

(22) PCT Filed: Oct. 5, 2023
(86) PCT No.: PCT/US2023/076039

§ 371 Date: Apr. 7, 2025

Related U.S. Application Data
(60) Provisional application No. 63/413,606, filed on Oct. 5, 2022.

(51) Int. Cl. A61K 38/57; A61K 35/28; A61P 19/02

(52) U.S. Cl. CPC A61K 38/57; A61K 35/28; A61P 19/02

(57) ABSTRACT

A pharmaceutical composition for use in promoting bone/cartilage regeneration, includes a PEDF-derived short peptide (PDSP); and mesenchymal stem cells (MSCs). The PDSP is 14-39 amino acids long. The PDSP has the amino-acid sequence selected from any one of SEQ ID NOs: 1-9. A concentration of the PDSP is about 10-200 μM.

### BACKGROUND OF INVENTION

### Field of the Invention

The present invention relates to PEDF-derived short peptides combined with mesenchymal stem cells and their uses in bone/cartilage regeneration.

### Background Art

Regenerative medicine is the medical field that development of innovative therapies focused on regrowth or repair of damaged or diseased cells or organs. The treatments include uses of therapeutic tissue-engineering products, platelet-rich plasma, autologous mesenchymal stem cells (MSCs), and/or allogenic biologics. One of the most common diseases investigated in regenerative therapies is osteoarthritis (OA).

Osteoarthritis (OA) is a common joint disorder affecting older people or young athletes. The disease can occur in any joint including hands, knees, hips, and feet. OA is characterized by pathologic changes in cartilages, subchondral bone, synovium, capsule, periarticular muscles, osteophyte formation, and low-grade inflammation. OA can lead to joint dysfunction, pain, stiffness, functional limitation, and loss of mobility. Most of the current therapies, such as pain reliever or anti-inflammatory drugs (NSAIDs), provide only symptomatic relief. At the late stages, surgical treatment, such as joint replacement surgery, knee osteotomy, or knee joint distraction, is often necessary to partially restore joint function.

However, those therapies are mostly designed to arrest structural damage progression, to reduce pain, and to improve mobility of joints, rather than promoting the regeneration of damaged cartilage. Stem cell therapy has provided new options for OA treatments. Mesenchymal stem cells (MSCs) can be isolated from multiple tissues, including adipose tissue (AT), umbilical cord (UC), infrapatellar fat pad (IPFP), Wharton's jelly, and the bone marrow (BM). MSCs are multipotent progenitor cells that are capable of self-renewal, multidirectional differentiations, and immune modulations. Several preclinical and clinical studies have used intra-articular MSC injection as a novel therapeutic strategy for the treatment of OA to reduce pain and improve joint structure/function.

### SUMMARY OF THE INVENTION

One aspect of the invention relates to pharmaceutical compositions for use in promoting bone/cartilage regeneration. A pharmaceutical composition for promoting bone/cartilage regeneration in accordance with one embodiment of the invention comprises a PEDF-derived short peptide (PDSP); and mesenchymal stem cells (MSCs). The PDSP is 14-39 amino acids long The PDSP has the amino-acid sequence selected from any one of SEQ ID NO: 1-9. A concentration of the PDSP is about 1-500 μM, preferably 10-200 μM, more preferably 10-100 μM. The pharmaceutical composition may further comprise one or more other active ingredients commonly used in treating osteoarthritis or repair of cartilage damages, such as analgesics (e.g., acetaminophen), nonsteroid anti-inflammatory drugs (NSAIDs, e.g., aspirin, ibuprofen, naproxen, and celecoxib), and corticosteroids. (see: Arthritis Foundation website).

Other aspects of the invention will become apparent with the following description and the enclosed drawings.

### BRIEF DESCRIPTION OF THE DRAWINGS

FIG. 1 shows a schematic of a treatment plan according to embodiments of the invention.

FIG. 2 shows treatment efficacies of PDSP with respect to the Osteoarthritis Research Society International (OARSI) scores.

FIGS. 3A & 3B show treatment efficacies of PDSP or combined with IPFP-MSC injection on Safranin O (FIG. 3A) and Hematoxylin and Eosin (H&E) (FIG. 3B) histological staining of the murine knee joints.

FIGS. 4A & 4B show treatment efficacies of PDSP or combined with IPFP-MSC injection on type II collagen (FIG. 4A) and X collagen (FIG. 4B) immunohistochemistry (IHC) staining of the murine knee joint.

FIG. 5A shows a protocol (timeline) for testing the effects of PDSP on human bone marrow MSC (hBM-MSC) proliferation and differentiation. FIG. 5B shows the staining of cells at day 14. FIG. 5C shows the total areas of the cells after 14 days. The total areas of the cells are functions of cell sizes, thereby serving as indicators for cell growth (proliferation). FIG. 5D shows Alcian Blue stained areas, which reflect the extents of chondrocyte differentiation.

### DETAILED DESCRIPTION

Human Pigment Epithelium-derived Factor (PEDF) is a secreted protein containing 418 amino acids, with a molecular weight of about 50 kDa. PEDF is a multifunctional protein with many biological functions (see e.g., U.S. Patent Application Publication No. 2010/0047212). Different peptide regions of the PEDF are found to be responsible for different functions. For example, a 34-mer fragment (residues 44-77 of PEDF) has been identified to have anti-angiogenic activity, while a 44-mer fragment (residues 78-121 of PEDF) has been identified to have neurotrophic properties.

U.S. Patent Application Publication No. 2010/0047212 discloses that PEDF can promote self-renewal of stem cells. U.S. Pat. No. 9,051,547 disclose that fragments of PEDF having 20-39 amino acids in length (residues 93-121 of PEDF) can promote stem cell proliferation and wound healing. U.S. Pat. No. 9,777,048 disclose that fragments of PEDF having 18-29 amino acids in length can promote chondrocyte proliferation, cartilage regeneration, and alleviation of pathology changes in joints associated with osteoarthritis. We recently found that the core peptide retaining these biological activities resides in the first 14 residues of the 29mer (SEQ ID NO:3). The 14mer core peptides are SEQ ID NO:8 (corresponding to residues 93-106 of human PEDF) and the corresponding mouse sequence (mo14mer; SEQ ID NO:9). One skilled in the art would appreciate that as long as a peptide includes one of these 14mer peptides, it should have the activity. Thus, a peptide for use with embodiments of the invention may be a 14mer, 15mer, 16mer, etc. These PEDF-derived short peptides are referred to as PDSP in this description. According to preferred embodiments of the invention, a PDSP may be 14-39 amino acids long. Exemplary PDSP used in this invention are listed in TABLE 1 below:

TABLE 1
Examples of PEDF derived short peptides (PDSP)

| | SEQ | | |
|---|---|---|---|
| | ID | | PEDF |
| Sequence | NO | Length | locations |
| LSVATALSALSLGAEQRTESIIHRALYYDLISSPDIHGT | 1 | 39 mer | 83-121 |
| ALSALSLGAEQRTESIIHRALYYDLISSPDIHGT | 2 | 34 mer | 88-121 |
| SLGAEQRTESIIHRALYYDLISSPDIHGT | 3 | 29 mer | 93-121 |
| SLGAEQRTESIIHRALYYDLISSP | 4 | 24 mer | 93-116 |
| SLGAEQRTESIIHRALYYDL | 5 | 20 mer | 93-112 |
| SLGAEHRTESVIHRALYYDLITNPDIHST | 6 | mo29 mer | |
| SLGAEHRTESVIHRALYYDL | 7 | mo20 mer | |
| SLGAEQRTESIIHR | 8 | 14 mer | 93-106 |
| SLGAEHRTESVIHR | 9 | mo14 mer | |

Embodiments of the invention relate to PDSP and their uses in the prevention and/or treatment of osteoarthritis (OA). The following specific examples will use results from clinical trial of the 29-mer (SEQ ID NO: 3) combined with MSC intra-articular injection to illustrate embodiments of the invention. While the clinical study used the 29mer (SEQ ID NO: 3), other PDSPs listed in the above table were also shown to have similar effects in preclinical studies. One skilled in the art would appreciate that the results of the 29mer PDSP are for illustration and are not intended to limit the scope of the invention.

The OA murine model is induced by anterior cruciate ligament (ACLT) surgery. In brief, a medial arthrotomy was performed. With the knee in full flexion, the patella was dislocated laterally, the anterior cruciate ligament (ACLT) was transected, and then the wounds were closed.

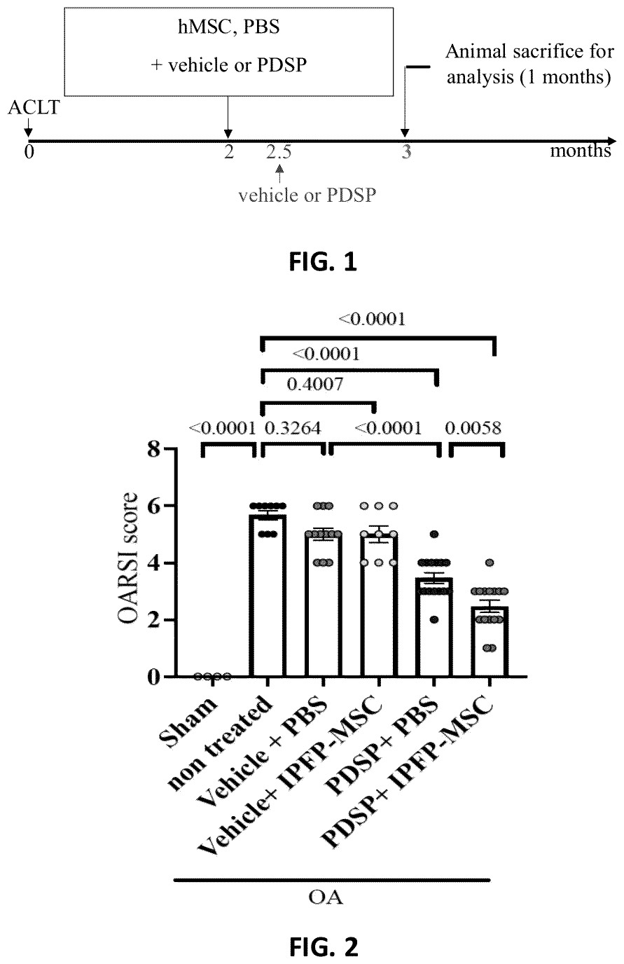

FIG. 1 shows an experimental protocol in accordance with one embodiment of the invention for testing the effects of PDSP. The ACLT surgery was performed on the unilateral knees of each mouse for 2 months prior to the treatments. Twenty mice were randomly divided into 5 groups: (i) non-treated group [n=3]; (ii) vehicle-treated group [n=4]; (iii) PDSP-treated group [n=5], (iv) vehicle combined with IPFP-MSC-treated group [n=3]; (v) PDSP combined with IPFP-MSC-treated group [n=5] in ACLT surgery induced OA groups, while the knees without surgery serve as sham-operated group [n=4]. At eight weeks after ACLT surgery (first injection), the knee joints of the non-treated, vehicle-treated, or PDSP-treated groups were intra-articular injected, respectively, with 0.01 mL PBS, vehicle, or PDSP, while those of the vehicle or PDSP combined with IPFP-MSC groups were injected with hMSCs in 0.01 mL. MSCs were intra-articular injected at the first treatment only (i.e., at 8 weeks). For the second treatments (two weeks after MSC injections or 10 weeks after ACLT surgery), those of the vehicle or PDSP combined with IPFP-MSC injection groups were injected with 0.01 mL vehicle or PDSP.

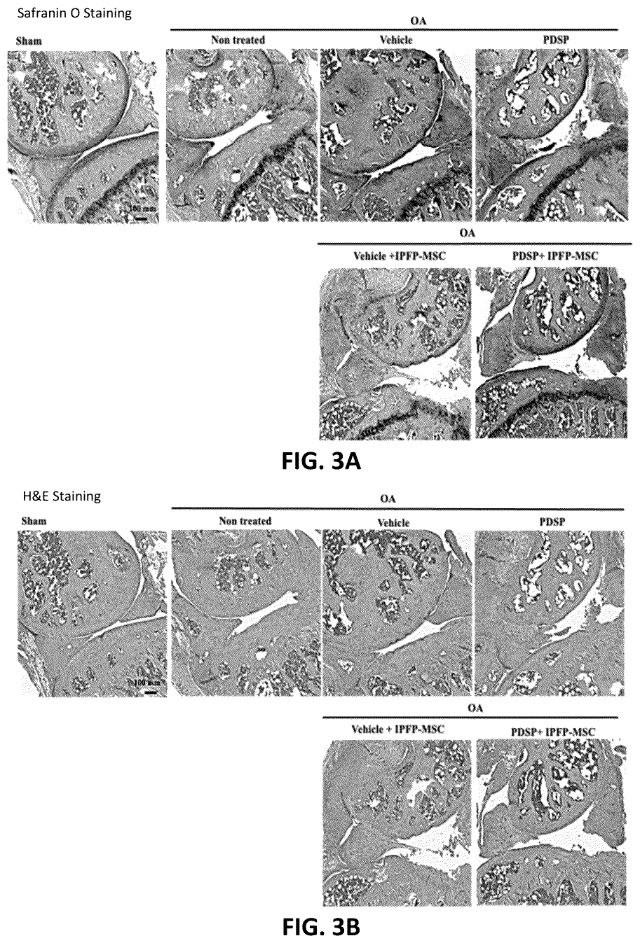

FIG. 2 shows the results of OARSI scores for various treatment groups. ORASI scores are used to evaluate the grades of cartilage degradations in the sham group, vehicle-treated, PDSP-treated, or vehicle/PDSP combined with infrapatellar fat pad-derived mesenchymal stem cell (IPFP-MSC)-treated groups in ACLT surgery induced OA groups. There was no significant difference in the ORASI scores between the non-treated and vehicle-treated OA groups (5.67±0.17 vs. 5±0.21; p=0.3264), suggesting that the vehicle alone did not show any therapeutic effects in OA groups in cartilage loss and surface irregularity. The OARSI scores were significantly decreased in the PDSP-treated OA groups, as compared with the non-treated or vehicle-treated OA groups (3.47±0.19 vs. 5.67±0.17 or 3.47 vs. 5±0.21; p<0.0001, p<0.0001). These results indicate that PDSP treatments could prevent cartilage loss and surface irregularity in the ACLT-surgery-indued OA model. In addition, the scores were also significantly decreased in the PDSP combined with IPFP-MSC injection-treated OA groups, as compared with the vehicle combined with IPFP-MSC injection or PDSP-treated groups (2.47±0.22 vs 5.67±0.17, p<0.0001; 2.47±0.22 vs 3.47±0.19, p=0.0058). These results indicate that the PDSP treatments could prevent the cartilage matrix loss and maintain surface regularity in the ACLT-surgery-indued OA model.

More importantly, PDSP combined with IPFP-MSC injection could enhance the efficiency of PDSP or IPFP-MSC alone. As shown in FIG. 2, vehicle-IPFP-MSC treatment alone has no effect (i.e., same as the vehicle treatment group). Thus, one would expect the combination treatment with PDSP and IPFP-MSC would produce an effect similar to that of PDSP treatment alone. Instead, the combination therapy produced an effect much better than PDSP treatment alone (see FIG. 2 and Table 2). The fact that the combination therapy produced an effect significantly greater than the sum of the two individual effects suggests a synergistic effect.

The results of ORASI scores are summarized in TABLE 2.

TABLE 2
The histologic scoring of murine OA by PDSP and IPFP-MSC treatments

| | | Animal | | | | | | |
|---|---|---|---|---|---|---|---|---|
| | | No. 1 | No. 2 | No. 3 | No. 4 | No. 5 | Average | SEM |
| Non surgery | Sham | 0, 0, 0 | 0, 0, 0 | 0, 0, 0 | 0, 0, 0 | | 0 | 0 |
| ACLT surgery | Non-treated | 6, 5, 6 | 6, 6, 6 | 5, 6, 5, | | | 5.67 | 0.17 |
| induced | Vehicle | 5, 4, 5 | 4, 6, 5 | 5, 5, 4 | 6, 5, 6 | | 5 | 0.21 |
| osteoarthritis | Vehicle + | 4, 5, 5 | 4, 6, 6 | 4, 6, 5 | | | 5 | 0.29 |
| | IPFP-MSC | | | | | | | |
| | PDSP | 3, 3, 4 | 4, 4, 3 | 3, 4, 3 | 3, 2, 3 | 4, 4, 5 | 3.47 | 0.19 |
| | PDSP + | 1, 2, 2 | 3, 2, 3 | 2, 3, 3 | 3, 3, 1 | 4, 2, 3 | 2.47 | 0.22 |
| | IPFP-MSC | | | | | | | |

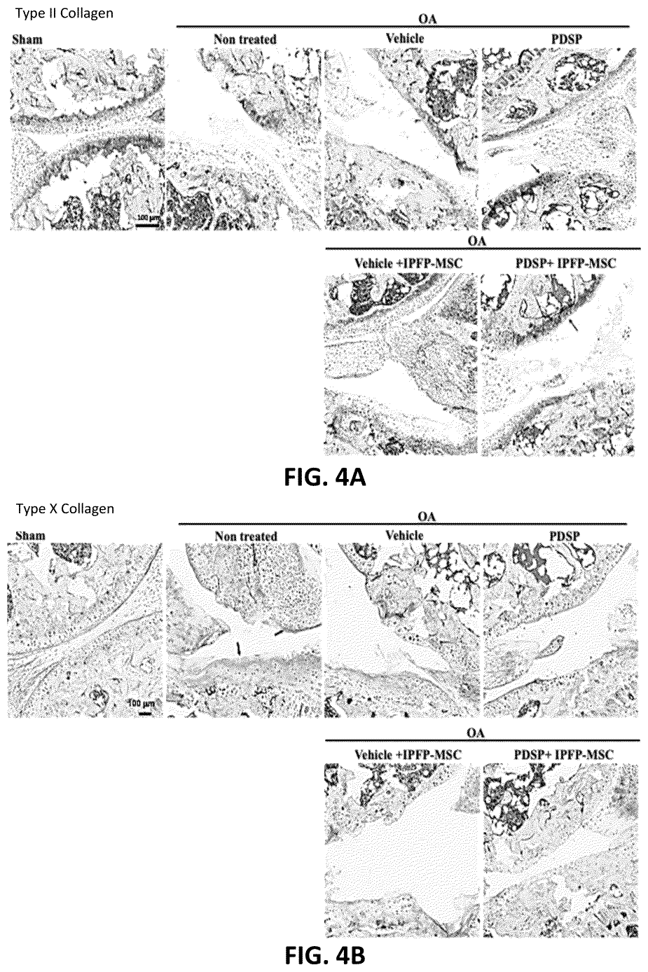

FIG. 3A and FIG. 3B show the results of safranin O staining and H&E staining, respectively. Compared to the sham operation groups, the results of safranin O and HE staining in the non-treated OA groups showed pathological changes in the joint, including cartilage matrix loss, surface irregularity, fibrillation or cleft, changes in cellularity, and loss of tidemark integrity. In contrast, the stains from the PDSP-treated and MSC combined with PDSP-treated groups show less cartilage loss and surface irregularity in the medial and lateral compartments. These results suggest that the PDSP treatments and PDSP combined with MSCs treatment could ameliorate the pathological changes in the joints.

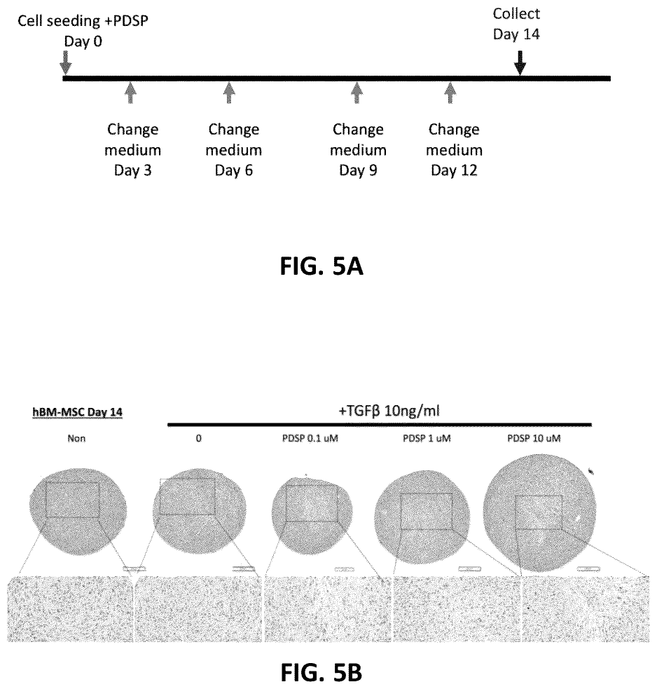

FIG. 4A and FIG. 4B show the results of type II and X immunohistochemistry (IHC) staining, respectively. Type II collagen is mainly found in cartilage, while type X collagen is found in bone and joint cartilage and is a potential marker for osteoarthritis. As shown in FIG. 4A, the expression of type II collagen was increased in the PDSP-treated and the MSC combined with PDSP-treated groups, as compared with the non-treated and vehicle-treated OA groups. Type X collagen signals were decreased in the PDSP-treated and the MSC combined with PDSP-treated groups (FIG. 4B). These results indicate that PDSP treatments could promote the regeneration of damaged cartilage or prevent cartilage loss.

The above results demonstrate the efficacies of PDSP combined with MSCs in the treatment of osteoarthritis. The studies used the safranin O/H&E/type II collagen/type X collagen staining to quantify cartilage structures and degradation on cartilage morphology and glycosaminoglycan contents in ACLT surgery induced osteoarthritis mouse model. The chondroprotective effects by PDSP combined with IPFP-MSC are more effective than treatments with vehicle, PDSP, IPFP-MSC, or vehicle combined with IPFP-MSC. Using a panel of histological staining and OARSI grading, the studies are able to predict the preclinical efficacies. Taken together, these in vivo studies provide essential insights and can be powerful evaluators of PDSP and PDSP combined with IPFP-MSC treatment efficacies. In view of the synergistic effect of combination therapy using PDSP and MSC, it would be advantageous to use the combination therapy to treat OA or promote chondrogenesis and prevent/repair cartilage damages.

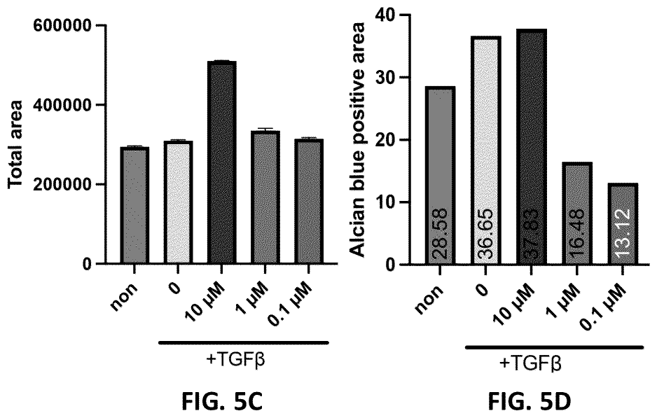

To shed light on possible mechanisms for the effects of PDSP chondrogenesis, further experiments were conducted to assess the effects of PDSP on MSC. The MSC used in this study are human bone marrow-derived mesenchymal stem cells (hBM-MSC) from Gibco (StemPro™ BM Mesenchymal Stem Cells). FIG. 5A shows a protocol (timeline) for evaluating PDSP on MSC chondrogenesis. Human bone marrow MSC spheroids were cultured with chondrogenic differentiation medium, with or without TGFβ1 and with or without PDSP. The media were changed every 3 days. On day 14, the cartilage spheroids were collected for evaluation. The various media compositions are as follows (the base medium is a high-glucose DMEM supplemented with 1×10⁻⁷M dexamethasone, 1×10⁻³M sodium pyruvate, 1.7×10⁻⁴M L-ascorbic acid-2-phosphate, 3.5×10⁻⁴M proline, 5 mL ITS+Premix in 500 mL medium, and 1% antibiotic):

| | Chondrogenic | Chondrogenic differentiation | | | |
|---|---|---|---|---|---|
| Culture | differentiation | medium (with TGFβ1 10 ng/ml) + | | | |
| Medium | medium without TGFβ1 | PDSP (μM) | | | |
| Group | non | 0 | 0.1 | 1 | 10 |

FIG. 5B shows results of Alcian Blue staining of cartilage spheroids from various groups. The total areas of the cartilage spheroids were as shown in FIG. 5C, and the Alcian blue positive areas are shown in FIG. 5D. As shown in FIG. 5C, PDSP (at 10 μM) was able to increase the cartilage spheroids size—i.e., promoting cell growth (cell proliferation). At the same time, PDSP (at 10 μM) was also able to promote chondrogenesis (i.e., differentiation of hBM-MSC into chondrocytes). These results suggest that the biological effects of PDSP may involve promotion of MSC proliferation and differentiation.

In accordance with embodiments of the invention, mesenchymal stem cells (MSCs) can be isolated from multiple tissues, including adipose tissue (AT), umbilical cord (UC), and the bone marrow (BM). MSCs are capable of differentiation into cartilages. The IPFP is a less invasive source of MSCs. If the amount MSCs is limited, amplification of MSCs may be required for clinical applications. MSCs isolated from bone marrow and adipose tissue are major sources for therapeutic applications in clinical trials.

While embodiments of the invention have been illustrated with a limited number of examples. One skilled in the art would appreciate that other modifications or variations are possible without departing from the scope of the invention. Therefore, the scope of protection should be limited by the accompanying claims.

1. A pharmaceutical composition for treating osteoarthritis or promoting bone/cartilage regeneration, comprising a PEDF-derived short peptide (PDSP); and mesenchymal stem cells (MSCs).

2. The pharmaceutical composition for treating osteoarthritis or promoting bone/cartilage regeneration according to claim 1, wherein the PDSP is 14-39 amino acids long.

3. The pharmaceutical composition for treating osteoarthritis or promoting bone/cartilage regeneration according to claim 1, wherein the PDSP has the amino-acid sequence selected from any one of SEQ ID NOs: 1-9.

4. The pharmaceutical composition for treating osteoarthritis or promoting bone/cartilage regeneration according to claim 1, wherein the PDSP has the amino-acid sequence of SEQ ID NO: 3.

5. The pharmaceutical composition for treating osteoarthritis or promoting bone/cartilage regeneration according to claim 1, wherein a concentration of the PDSP is about 10-200 μM.

6. The pharmaceutical composition for treating osteoarthritis or promoting bone/cartilage regeneration according to claim 1, wherein the mesenchymal stem cells are from adipose tissue, umbilical cord, bone marrow, or infrapatellar fat pad.

7. The pharmaceutical composition for treating osteoarthritis or promoting bone/cartilage regeneration according to claim 1, wherein the pharmaceutical composition further comprises one or more other active ingredients.

8. The pharmaceutical composition for treating osteoarthritis or promoting bone/cartilage regeneration according to claim 7, wherein the one or more other active ingredients comprise an analgesic, a nonsteroid anti-inflammatory drug, or a corticosteroid.

9. The pharmaceutical composition for treating osteoarthritis or promoting bone/cartilage regeneration according to claim 2, wherein a concentration of the PDSP is about 10-200 μM.

10. The pharmaceutical composition for treating osteoarthritis or promoting bone/cartilage regeneration according to claim 3, wherein a concentration of the PDSP is about 10-200 μM.

11. The pharmaceutical composition for treating osteoarthritis or promoting bone/cartilage regeneration according to claim 4, wherein a concentration of the PDSP is about 10-200 μM.

12. The pharmaceutical composition for treating osteoarthritis or promoting bone/cartilage regeneration according to claim 2, wherein the mesenchymal stem cells are from adipose tissue, umbilical cord, bone marrow, or infrapatellar fat pad.

13. The pharmaceutical composition for treating osteoarthritis or promoting bone/cartilage regeneration according to claim 3, wherein the mesenchymal stem cells are from adipose tissue, umbilical cord, bone marrow, or infrapatellar fat pad.

14. The pharmaceutical composition for treating osteoarthritis or promoting bone/cartilage regeneration according to claim 4, wherein the mesenchymal stem cells are from adipose tissue, umbilical cord, bone marrow, or infrapatellar fat pad.

15. The pharmaceutical composition for treating osteoarthritis or promoting bone/cartilage regeneration according to claim 5, wherein the mesenchymal stem cells are from adipose tissue, umbilical cord, bone marrow, or infrapatellar fat pad.


SEQUENCE LISTING (9 sequences)

SEQ ID NO: 1 - protein, 39 aa, Homo sapiens
LSVATALSALSLGAEQRTESIIHRALYYDLISSPDIHGT

SEQ ID NO: 2 - protein, 34 aa, Homo sapiens
ALSALSLGAEQRTESIIHRALYYDLISSPDIHGT

SEQ ID NO: 3 - protein, 29 aa, Homo sapiens
SLGAEQRTESIIHRALYYDLISSPDIHGT

SEQ ID NO: 4 - protein, 24 aa, Homo sapiens
SLGAEQRTESIIHRALYYDLISSP

SEQ ID NO: 5 - protein, 20 aa, Homo sapiens
SLGAEQRTESIIHRALYYDL

SEQ ID NO: 6 - protein, 29 aa, Mus musculus
SLGAEHRTESVIHRALYYDLITNPDIHST

SEQ ID NO: 7 - protein, 20 aa, Mus musculus
SLGAEHRTESVIHRALYYDL

SEQ ID NO: 8 - protein, 14 aa, Homo sapiens
SLGAEQRTESIIHR

SEQ ID NO: 9 - protein, 14 aa, Mus musculus
SLGAEHRTESVIHR